(19) United States
(12) Patent Application Publication
(10) Pub. No.: US 2006/0095021 A1
(43) Pub. Date: May 4, 2006

(54) Introduction of agent with medical device

(76) Inventors: Jesus Wilfredo Casas-Bejar, Brooklyn Park, MN (US); Laura J. Christoferson, Ramsey, MN (US); Adam A. Blakstvedt, Big Lake, MN (US)

Correspondence Address: MEDTRONIC, INC., 710 MEDTRONIC PARK, MINNEAPOLIS, MN, 55432-9924, US

(21) Appl. No.: 10/979,774

(22) Filed: Nov. 2, 2004

(51) Int. Cl. A61K 9/22

(52) U.S. Cl. 604/892.1; 604/95.03

(57) ABSTRACT

The disclosure is directed to apparatus and techniques that deliver an antibiotic or loosening agent to a patient by diffusion. An element of the medical device deployed proximate to the living cells includes a diffusible material, which comprises a lumen. An agent introduced into the lumen diffuses through the diffusible material to the living cells or tissues. The invention can be applied to medical devices that are placed partially inside a patient, as well as those that are fully implanted. Some embodiments support moving the internal element of a medical device proximate to targeted cells, such as a tumor, and administering an antibiotic agent to the targeted cells by diffusion. The disclosure also encompasses a testing system that helps test and develop apparatus and techniques for delivering an agent by diffusion.

### TECHNICAL FIELD

The invention relates to medical devices, and in particular, medical devices that are deployed in whole or in part inside a human or animal body.

### BACKGROUND

Some medical devices, such as catheters, can be inserted into a human or animal body and remain inserted for days, weeks or months. Other medical devices, such as implantable pacemakers, defibrillators or drug pumps, may be implanted in the body and are expected to remain implanted for years. When a medical device is inserted into or implanted in a body, there is a risk of infection associated with the inserted or implanted device. Bacteria such as staphylococcus aureus and staphylococcus epidermidis, for example, can cause serious health concerns. Bacteria such as these can colonize the device at any time following implantation, sometimes within a matter of days, and produce an infection and be a source for serious health concerns such as generalized infection or septicemia.

When a patient experiences an infection, conventional procedure may be to treat the patient with antibiotics. Conventional antibiotic treatments deliver the antibiotic systemically, such as by a bolus injection into the bloodstream or by oral ingestion of antibiotic pills. It is not uncommon, however, for such systemic treatment to fail to kill the bacteria responsible for the infection. In many cases, bacteria form a biofilm that protects the infection from the antibiotics. In other cases, the patient's own body develops an encapsulation around elements of the device inside the body, shielding the infection from the antibiotics.

Despite systemic therapy, in some cases the infection persists, and extraction of the device is indicated. In many circumstances, extraction is an undesirable option. Explantation of a fully implanted medical device, for example, is inconvenient, expensive, and may cause additional risks to the patient. One of the risks associated with extraction of a device is that the patient over time forms tissues that can make it difficult for the surgeon to obtain access to the device. In particular, such tissues can resist extraction of the device.

Some patients experience medical problems arising not from an infection by foreign cells, but rather from their own cells. When the patient's own cells turn cancerous, for example, the consequences can be serious. Various cancer treatments, such as surgery, radiation and chemotherapy, can have diverse rates of success and diverse side effects for the patient.

### SUMMARY

In general, the invention is directed to techniques in which an antibiotic agent is delivered by diffusion to living cells in a patient, to bring the antibiotic agent to an infection or tumor. In addition, in some embodiments, the invention is directed to techniques for delivery of a loosening agent to restraining tissues, thereby aiding in extraction of an implanted medical device. The invention is also directed to devices for carrying out such techniques. The antibiotic or loosening agent is introduced into a lumen in a diffusible material, and the agent diffuses through the diffusible material. When an antibiotic agent is delivered in this way, the antibiotic agent has a therapeutic effect, such as killing the living cells or inhibiting their growth, while reducing adverse impacts upon healthy tissues. When a loosening agent is delivered in this way, the loosening agent helps disengage an implanted medical device from restraining tissues, which aids in the surgical removal of the implanted device. The invention also includes systems that help with the development and testing of the apparatus and the techniques.

One application of the invention addresses infections that often become associated with medical devices placed wholly or partially inside a patient. In accordance with the invention, the medical device includes a diffusible material, and the diffusible material comprises one or more lumens. An antibiotic or loosening agent is introduced into a port of the medical device, which is in fluid communication with the lumens, and as a consequence, the antibiotic or loosening agent is introduced into the lumens. The diffusible material is configured to pass the antibiotic or loosening agent by diffusion. In other words, the antibiotic or loosening agent is configured to move through the diffusible material by diffusion to the tissues or living cells that are nearby. Examples of diffusible materials include biocompatible silicone, polyethylene and polyurethane.

In the case of a bacterial infection that is in contact with the medical device, for example, an antibiotic agent diffuses to the infection site and destroys the infection or inhibits its growth. Delivery of the antibiotic agent by diffusion can allow the antibiotic agent to overcome obstacles such as biofilm or tissue encapsulation. Obstacles such as these can hinder the effectiveness of an antibiotic agent administered in other ways, such as by a bolus injection into the bloodstream or by oral ingestion of antibiotic pills. In the case of an antibiotic agent delivered by diffusion, the obstacles can be bypassed or broken down effectively.

In this way, the delivery of the antibiotic agent is more targeted toward particular living cells than a bolus injection or an oral ingestion would be. In addition, healthy cells proximate to the medical device would, in many cases, be able to handle the antibiotic agent without adverse effects.

The invention can be used to deliver a variety of antibiotic agents. Examples of antibiotic agents include antibacterial agents, cytotoxic agents, or Reactive Oxygen Species (ROS) agents. One ROS agent, hydrogen peroxide, is believed to have many desirable qualities as an antibiotic agent. Hydrogen peroxide diffuses well, is effective against a range of infections and tumors, and is usually well tolerated by healthy tissues.

Because the delivery of the antibiotic agent can be targeted toward particular living cells, the invention can be employed to move a medical device proximate to target cells, and administer the antibiotic agent to those target cells. A patient may have a tumor, or example, or a localized infection. In such as case, a medical device can be moved proximate to the tumor or infection. In particular, a medical device with at least a portion made of diffusible material can be moved proximate to the tumor or infection. The diffusible material comprises one or more lumens. When an antibiotic agent is introduced into the lumens, it diffuses through the diffusible material to the target cells.

In the case of an implanted medical device that has become restrained by tissues, the restraining tissues can be an impediment to surgical removal of the medical device. Prior to surgical removal, a loosening agent can be introduced into a port of the implanted medical device. The loosening agent passes by diffusion through the diffusible material and acts upon the restraining tissues by dissolving or otherwise loosening the tissues from the implanted device. As a result of delivery of the loosening agent, the medical device can be more easily removed during the surgical removal procedure. The invention can be used to deliver a variety of loosening agents. Examples of loosening agents include ROS agents such as hydrogen peroxide.

In one embodiment, the invention is directed to a medical device comprising an element configured to be deployed proximate to living cells in a patient, and a port configured to receive an antibiotic agent. The element comprises a diffusible material configured to be in contact with the living cells, and also comprises at least one lumen in fluid communication with the port. The diffusible material is configured to diffuse the antibiotic agent in the lumen to the living cells. In the case of an implantable medical device, the “element” can be the entire medical device. The invention also encompasses embodiments that include internal and external elements, as well as jacketing devices that jacket at least a part of a medical device that is configured to be deployed proximate to living cells in the patient.

In another embodiment, the invention presents a method comprising introducing an antibiotic agent into a port of a medical device that includes at least one element configured to be deployed proximate to living cells in a patient. The element comprises a diffusible material configured to be in contact with the living cells, and the diffusible material comprises at least one lumen in fluid communication with the port. The introduced antibiotic agent is configured to diffuse from the lumen to the living cells.

In a further embodiment, the invention is directed to a device comprising an elongated primary core having an axis surrounded by a diffusible material having an exterior surface, a first lumen in the diffusible material configured to conduct an antibiotic agent in a first direction, and a second lumen in the diffusible material configured to conduct the antibiotic agent in a second direction. The diffusible material is configured to diffuse the antibiotic agent from at least one of the lumens to the exterior surface of the device. This embodiment of the invention encompasses a variety of elongated medical devices, such as catheters and endoscopes.

In an additional embodiment, the invention is directed to a device comprising a port configured to receive an antibiotic agent and at least one lumen in fluid communication with the port. The device, which may be called a “jacketing device,” comprises a diffusible material, and is configured to jacket at least a part of a medical device. The diffusible material is configured to diffuse the antibiotic agent in the lumen to the living cells. An advantage of a jacketing device is that it can be coupled to a pre-existing medical device that lacks diffusible material, lumens or a port.

In another embodiment, the invention is directed to a system comprising a test platform configured to support an indicator, and an experimental tube made at least in part of diffusible material deployed in the test platform proximate to the indicator. The test platform can be, for example, a culture plate containing a growth medium, and the indicator can be a test cell culture on the medium. The experimental tube comprises a lumen configured to receive a test agent, which can be an antibiotic agent or a loosening agent. This “test system” supports testing of kinds of diffusible materials, geometries of diffusible materials, and the effectiveness of agents against particular living cells or tissues. Information obtained in such in vitro testing helps in the development of devices deployed in vivo.

In an added embodiment, the invention presents a method comprising introducing a test agent into a lumen of an experimental tube deployed in a test platform. The experimental tube is deployed proximate to an indicator, and the experimental tube is made at least in part of diffusible material. The invention also includes observing an effect upon the indicator.

The invention also encompasses embodiments in which a loosening agent can be delivered that helps disengage a medical device from restraining tissues such as collagen. Loosening agents can be, but need not be, antibiotic agents, and vice versa. An example of one loosening agent is hydrogen peroxide, which can also serve as an antibiotic agent.

The various embodiments of the invention may bring about one or more advantages. The invention provides apparatus and methods by which medical devices that are wholly or partially deployed inside a patient for extended periods of time can be protected from infection. The antibiotic agents are targeted around one or more specific sites, in contrast to an antibiotic agent ingested in pill form or injected into the bloodstream. Side effects are expected to be low, and healthy tissue is often not adversely affected.

The invention supports a variety of applications. The invention supports prevention of infections proximate to device elements deployed inside the body of a patient, such as implantable pulse generators, pumps, sensors, leads and catheters, as well as therapy to address infections that have developed proximate to the elements. The invention also supports targeting therapy to particular target cells. A medical device may be deliberately moved proximate to target cells, and antibiotic agents may be administered by diffusion to those target cells.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual perspective diagram illustrating an exemplary medical device that illustrates the features of the invention.

FIG. 2 is a conceptual perspective diagram of the medical device of FIG. 1, illustrating additional features of the invention.

FIG. 3 is a conceptual perspective diagram of a medical device configured to jacket another medical device according to an embodiment of the invention.

FIG. 4 is a cutaway view of another exemplary medical device that illustrates features of the invention.

FIG. 5 is a cross-sectional view of a lumen of the device shown in FIG. 4, illustrating diffusion of antibiotic agents through a diffusible material.

FIG. 6 is a conceptual perspective diagram of a further exemplary medical device, illustrating a medical device with internal and external elements.

FIG. 7 is a conceptual perspective diagram of an additional illustrative medical device having internal and external elements, with the internal elements including a balloon.

FIG. 8 is a block diagram illustrating an exemplary system suitable for implementation of the techniques of the invention.

FIG. 9 is a flow diagram illustrating an exemplary procedure employing techniques of the invention.

FIG. 10 is a conceptual diagram illustrating an in vitro testing system according to the principles of the invention.

### DETAILED DESCRIPTION

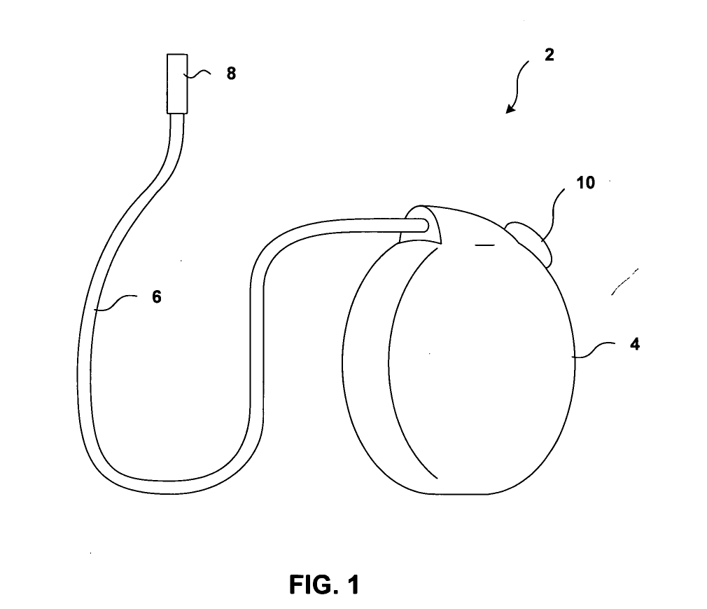

FIG. 1 is a conceptual diagram illustrating a medical device 2 according to an embodiment of the invention. In the embodiment depicted in FIG. 1, medical device 2 is a fully implantable medical device, that is, a medical device that is implanted inside the body of a living person or animal. Medical device 2 could include any of several implantable medical devices, such as a pacemaker, implantable cardioverter-defibrillator, implantable drug pump, implantable neurostimulator, patient monitor, physiological sensor, lead and the like.

The invention is not limited to medical devices that are fully implantable. As will be discussed below, the invention also includes embodiments in which at least a portion of the device is deployed internally proximate to living cells in a patient. Other components of the device can be external to the patient or otherwise remote from the living cells.

For purposes of illustration, medical device 2 includes a body 4 and an extension 6. The functions of body 4 and extension 6 vary from device to device. When medical device 2 is designed to supply pacing therapy to a heart, for example, body 4 represents the pacemaker and extension 6 represents one or more leads that extend to the heart, and the distal end 8 of extension 6 includes one or more pacing electrodes. When medical device 2 is a drug pump that delivers drugs to the patient, body 4 represents a pump and a reservoir for the drugs being delivered. For a drug pump, extension 6 represents one or more catheters that administer the drugs to the cells, with distal end 8 being deployed proximate to the cells of concern.

Medical device 2 includes a port 10 configured to receive an antibiotic agent or loosening agent. For simplicity, the invention will be initially described in the context of an antibiotic agent.

Port 10 may comprise, for example, a self-sealing membrane. When implanted in a living body, the antibiotic agent may be introduced into medical device 2 by a hypodermic needle that penetrates the skin and enters port 10. The antibiotic agent may be stored in a reservoir (not shown in FIG. 1) coupled to port 10.

The antibiotic agent may comprise one or more antibiotic agents, including an antibacterial agent, an antimicrobial agent, an antiproliferative agent, a cytotoxic agent, or a Reactive Oxygen Species (ROS) agent such as hydrogen peroxide. These categories of antibiotic agents do not necessarily comprise an exclusive list of antibiotic agents, and the categories are not necessarily exclusive of one another. Some antibiotic agents, including some ROS agents, have both antibacterial and antitumoral applications, for example. The function of the antibiotic agent is to affect living cells that are or could be harmful to the patient. In some cases, the living cells comprise microorganisms such as bacteria that infect the patient. In other cases, the living cells comprise the patient's own cells, which have transformed into cancerous cells. As used herein “antibiotic agent” includes agents that kill living cells, such as microorganisms or cancer cells. “Antibiotic agent” also includes agents that impede the growth or spread of living cells or that are otherwise employed to provide chemotherapeutic treatment of diseases or infections.

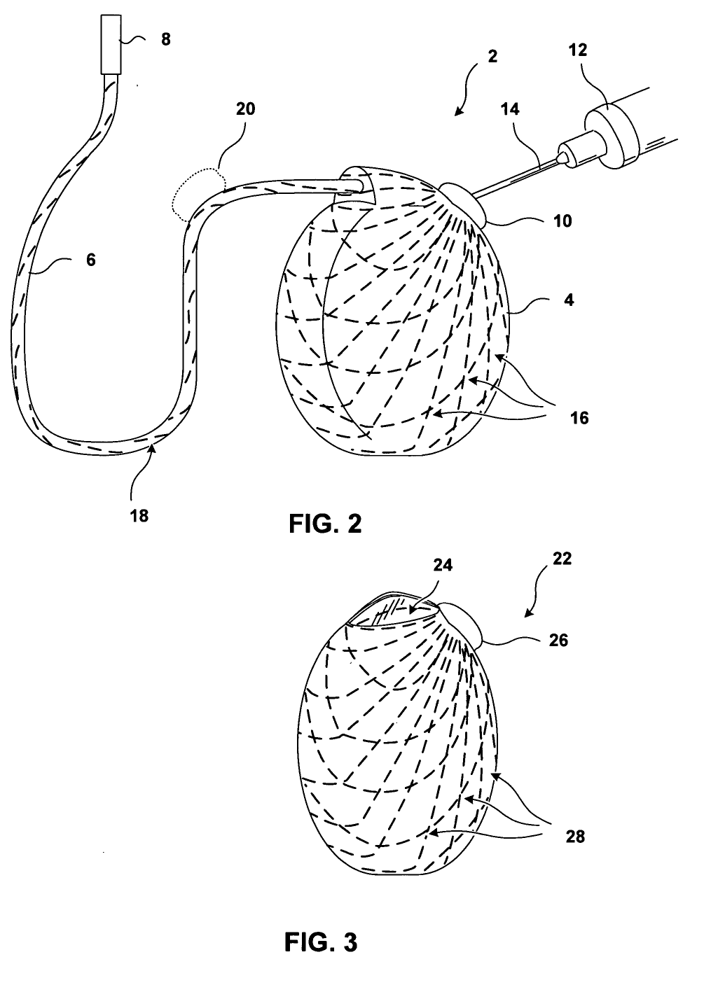

FIG. 2 shows medical device 2 with a syringe 12 injecting an antibiotic agent into port 10. Medical personnel inserts needle 14 through the skin of the patient and into port 10, and injects the antibiotic agent into port 10. Port 10 may include a self-sealing membrane to prevent leakage. The body of syringe 12 comprises a reservoir for the antibiotic agent, and in some embodiments, antibiotic agent is also stored in an internal reservoir (not shown in FIG. 2) coupled to port 10.

The antibiotic agent injected into port 10 circulates through a plurality of lumens 16 that are in fluid communication with port 10. Lumens 16 can be deployed as individual lumens that do not intersect or interact with one another. Lumens 16 can also be arranged in an array of lumens, as indicated by the dashed lines in FIG. 2. Lumens 16 comprise one or more passageways that are configured to receive the antibiotic agent loaded into medical device 2 via port 10, and to distribute the antibiotic agent in or around medical device 2. In the embodiment of the invention depicted in FIG. 2, lumens 16 interconnect and surround medical device 2 in a mesh-like configuration.

Medical device 2 includes a diffusible material, which comprises one or more lumens 16. In other words, lumens 16 represent fluid passageways in the diffusible material. The diffusible material may comprise any material that permits antibiotic agents in lumens 16 to diffuse to living cells proximate to medical device 2. It has been discovered that certain antibiotic agents, such as hydrogen peroxide, can diffuse through polymers or elastomers such as biocompatible silicone or polyurethane, without special modification to the silicone or polyurethane.

Hydrogen peroxide offers many benefits as an antibiotic agent: it diffuses readily, maintains potency after diffusion, and is effective in killing aerobic and anaerobic bacteria. Hydrogen peroxide is freely miscible with water and can cross cell membranes readily. Importantly, most healthy cell tissues can remove hydrogen peroxide without adverse effect.

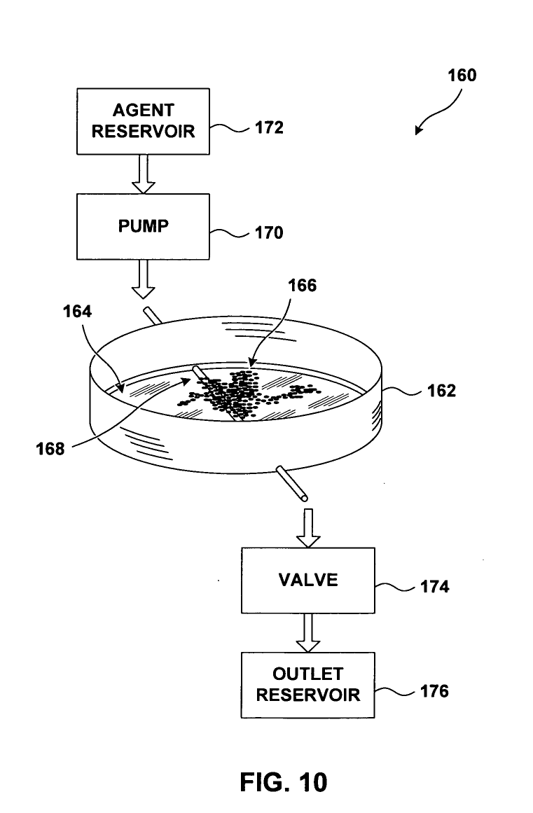

In an in vitro test using an apparatus similar to that depicted in FIG. 10, a thirty percent concentration hydrogen peroxide solution demonstrated substantial effectiveness against staphylococcus epidermidis, a common source of device-associated infections. Diffusing through polyethylene 80 (PE80A) tubing, the hydrogen peroxide demonstrated a zone of inhibition of bacterial growth in excess of eighty millimeters from the site of diffusion. Lower concentrations of hydrogen peroxide demonstrated smaller zones of inhibition. A 0.3 percent hydrogen peroxide concentration, for example, demonstrated a zone of inhibition of bacterial growth of about twenty-five millimeters in vitro. An advantage of an antibiotic agent with a larger zone of inhibition is that the antibiotic agent can be effective in a device that has lumens more widely spaced from one another.

The invention supports use of diffusive antibiotic agents ion addition to hydrogen peroxide. The invention also supports the use of diffusible materials in addition to biocompatible silicone, polyethylene or polyurethane.

In general, the antibiotic agents in lumens 16 follow a concentration gradient, moving from a region of high antibiotic agent concentration in lumens 16 to a region of low antibiotic agent concentration. As a result, the antibiotic agents in lumens 16 generally diffuse to the exterior surface of medical device 2, where medical device 2 interfaces with living cells.

In the embodiment shown in FIG. 2, the diffusible material may be in the form of a covering that encloses the housing of medical device 2, and may be incorporated into medical device 2 during the construction of medical device 2. The diffusible material may be, for example, a silicone covering, in which lumens 16 have been created by a net that is removed after the coating is completed. Alternatively, the diffusible material may be constructed as component that can be added to medical device 2 after the construction of medical device 2, as depicted in FIG. 3.

It has been discovered that certain antibiotic agents diffuse through diffusible materials such as biocompatible silicone. In general, the antibiotic agents follow a concentration gradient, moving from a region of high antibiotic agent concentration in lumens 16 to a region of low antibiotic agent concentration. Biocompatible silicone is not the only material that is diffusible, and the invention encompasses embodiments that include other diffusible materials. Various elastomeric materials and polymers may also support diffusion of antibiotic agents. Polyurethane is one example of another diffusible material that can support diffusion of antibiotic agents.

Antibiotic agents diffusing through the diffusible material kill or otherwise affect harmful organisms that are in contact with medical device 2. These harmful organisms, which may be shielded from antibiotic agents applied externally, are generally susceptible to antibiotic agents that diffuse through the diffusible material. In contrast to delivery of antibiotic agents in a conventional way, such as by bolus injection, diffusion of the antibiotic agents through the diffusible material of a device can be localized near the device.

In some embodiments of the invention, a single medical device 2 can have more than one lumen or lumen array. In FIG. 2, for example, first lumen array 16 may cover medical device body 4, and second lumen array 18 may cover extension 6. FIG. 2 shows an optional second port 20 that receives an antibiotic agent, which circulates into second lumen array 18.

In further embodiments of the invention, the port may be internal to the medical device. When medical device 2 is a drug pump, for example, body 4 may include a first reservoir for holding drugs to be pumped to the body via distal end 8, and a second reservoir dedicated to antibiotic agents to be pumped into lumens 16. In such an embodiment, the port that couples the reservoir to the lumens can be internal to the device.

FIG. 2 shows loading of an antibiotic agent into device 2 without creation of an incision to obtain access to medical device 2. The invention also supports obtaining access to implantable medical device 2 via surgery. Making an incision to obtain access to medical device 2 may be desirable when, for example, it is desired that an outlet reservoir be coupled to port 10, as described below.

FIG. 3 shows the invention embodied as jacketing device 22 made, at least in part, of diffusible material that jackets a medical device. Jacketing device 22 surrounds or jackets at least a part of an implanted medical device or an internal element of a medical device. Such a jacketing device is ordinarily customized to a particular medical device. Jacketing device 22 is configured to jacket body 4 of the device shown in FIGS. 1 and 2.

In the embodiment shown in FIG. 3, jacketing device 22 is a bag-like device made of an elastomeric material that can be stretched. Jacketing device 22 is slipped over body 4 by inserting body 4 into opening 24. The elastomer of jacketing device 22 stretches to allow jacketing device 22 to receive body 4, and contracts to hold jacketing device 22 in place with respect to medical device 2.

Jacketing device 22 includes a port 26, which is coupled to a lumen array 28. Port 26 and lumen array 28 may be comparable to port 10 and lumen array 16 shown in FIG. 2. Jacketing device 22 may be a bag-like structure, as depicted in FIG. 3. In some embodiments, jacketing device 22 can be embodied as a net-like device.

A health care provider may jacket an implantable medical device with a jacketing or an internal element of a medical device prior to implantation. In the case of a medical device that has internal and external elements, the health care provider may jacket the internal element prior to introducing the internal element into the body of the patient. When an antibiotic agent is introduced into lumen array 28 via port 26, the antibiotic agent diffuses through the diffusible material to kill or otherwise affect harmful organisms that are in contact with jacketing device 22.

Medical device 2 or jacketing device 22 can also be used to deliver a loosening agent configured to disengage medical device 2 from restraining tissues. Restraining tissues are substances that adhere to, surround, encapsulate, or otherwise interfere with surgical removal of medical device 2. When a device is implanted in a patient, the patient forms encapsulating tissue that surrounds, and sometimes adheres to, the implanted device. In a typical patient, inflammatory cells surround the device shortly after implantation, and within weeks, fibroblasts and macrophages appear around the implanted device, followed by collagen deposition. In some cases, the tissue around the device can calcify. When the implanted device fails or is subject to removal for other reasons, restraining tissue such as collagen can impede surgical removal of the device. The restraining tissues can make it difficult for the surgeon to obtain access to the device, and can also resist extraction. The loosening agent breaks down, dissolves, dislodges, or otherwise loosens the restraining tissue from the implanted device, facilitating access and extraction.

Hydrogen peroxide is one example of a loosening agent. Hydrogen peroxide oxidizes collagen, and inhibits calcification. As a result, introduction of hydrogen peroxide into medical device 2 or jacketing device 22 can serve as both a loosening agent and as an antibiotic agent.

Other agents, such as pharmaceuticals or enzymes, can also serve as loosening agents. The loosening agents need not be antibiotic agents, and need not rely upon oxidation to loosen the implanted device from the restraining tissue. An enzyme such as collagenase, for example, can promote degradation of collagen by catalysis. Various loosening agents can be formulated to diffuse through the diffusible material to the restraining tissues, and an apparatus similar to that shown in FIG. 10 can be used to test in vitro how well a particular loosening agent diffuses through a particular diffusible material.

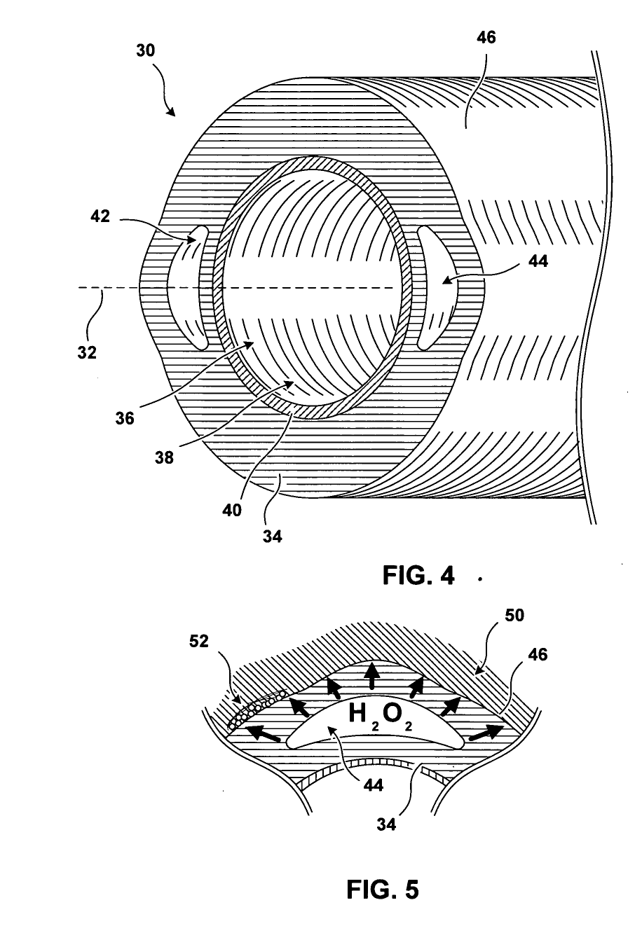

FIG. 4 is a cutaway view of another exemplary medical device that can carry out the invention. FIG. 4 shows an elongated tube 30 configured to be deployed proximate to living cells. For purposes of illustration, tube 30 has an axis 32. Body 34, which surrounds axis 32, is made of a diffusible material such as biocompatible silicone. Body 34 defines a primary core 36, which is substantially coincident with axis 32.

In FIG. 4, primary core 36 comprises a central passage 38 optionally surrounded by a lining 40. Lining 40 prevents the fluids in central passage 38 from diffusing through diffusible body 32. Lining 40 further inhibits diffusion of antibiotic or loosening agents to the primary core. Lining 40 may be constructed from any material that inhibits diffusion, including a variety of biocompatible polymers or coatings. Lining 40 may be of any thickness.

In the example of FIG. 4, tube 30 can be a catheter such as a catheter coupled to an implantable drug pump. In other embodiments, however, primary core 36 may be configured for other purposes. In an embodiment in which tube 30 is a lead coupled to a pacemaker or a neurostimulator, for example, primary core 36 houses the electrical leads that couple the pace-sense electrodes to the pacemaker sensing and stimulation circuitry. Tube 30 can also be adapted to other medical apparatus, such as a probe or endoscope.

Diffusible body 34 further includes lumens 42, 44 that are configured to conduct an antibiotic or loosening agent from a port and to permit the agent to diffuse from lumens 42, 44 to the living cells. FIG. 4 depicts tube 30 as having two lumens, but the invention is not limited to the particular embodiment shown. There may be any number of lumens, and the lumens may be deployed along body 34 in any fashion.

In a typical embodiment of a medical device that can carry out the invention, the antibiotic or loosening agent can circulate through the medical device. Accordingly, tube 30 may be coupled to the port in such a manner that lumens 42 and 44 are in fluid communication with one another and comprise a single passageway in which the agent can circulate. In lumen 42, an agent may flow in one direction, and the agent may flow in the opposite direction in lumen 44. Lumen 42 may be an afferent lumen, for example, in closer proximity to a port at which the agent is introduced. Lumen 44 may be an efferent lumen, in closer proximity to an outlet port. Lumens 42 and 44 may join one another at a site such as a distal end of a medical device. In such an implementation, a circulating agent would flow in one direction through afferent lumen 42 up to the distal end of the device, and would flow in a different direction away from the distal end via efferent lumen 44.

An agent introduced into lumens 42 and 44 diffuses through diffusible body 34. Lining 40 inhibits diffusion into primary core 36. Accordingly, diffusion generally result in the agent diffusing to the exterior surface 46 of tube 30, where living cells or restraining tissues, or both, come in contact with tube 30.

FIG. 5 is a cross-sectional view of exemplary lumen 44 from tube 30 shown in FIG. 4. In the example of FIG. 5, the antibiotic or loosening agent in lumen 44 is hydrogen peroxide. As shown in FIG. 5, hydrogen peroxide diffuses outward from lumen 44 through diffusible body 34 toward exterior surface 46, at which point the hydrogen peroxide comes in contact with living cells.

Some of the cells that receive hydrogen peroxide are the cells 52 of the patient's body. In the ordinary implementation of the invention, the amount or concentration of hydrogen peroxide would pose little danger to the patient's own cells 52. In general, certain well-vascularized tissues are not likely to be affected by hydrogen peroxide concentrations, or concentrations of other ROS agents, that are bactericidal. Catalases and other physiological antioxidant or oxidant scavengers present in normal tissue generally protect the normal tissue from adverse effects. It is noted that cardiac muscle may exhibit an inferior ability to remove hydrogen peroxide, so use of hydrogen peroxide as an antibiotic agent might be avoided when tube 30 is deployed proximate to cardiac muscle.

FIG. 5 depicts a developing infection 52, i.e., a colony of bacteria such as staphylococcus aureus, on surface 46 of diffusible body 34. Infections by organisms such as staphylococcus aureus can cause serious health concerns. Conventional administration of antibiotics may be ineffective in destroying the infection, for any of a number of reasons. As described above, the body naturally forms restraining tissue around or proximate to many implanted devices, and the restraining tissue can shield the infection from antibiotics. In addition, some bacteria form a biofilm that protects the bacteria from antibiotics.

Neither restraining tissue nor biofilm protects infection 52 from the antibiotic agent diffusing through diffusible body 34. When present, restraining tissue is not interposed between bacteria 52 and diffusible body 34. A biofilm, even if interposed between bacteria 52 and diffusible body 34, provides no protection. Most biofilms have been found to exhibit patches of cell aggregates, rather than monolayers, that are interspersed throughout an exopolysaccharide matrix that varies in density. As a result, open areas in the biofilms are created, and the biofilms are generally permeable to oxidative agents such as hydrogen peroxide. Hydrogen peroxide permeating through a biofilm would destroy bacteria 52. In this way, hydrogen peroxide diffusing outward from lumen 44 through the diffusible material of body 34 contacts and destroys infection 52 by processes such as oxidation, peroxidation and decarboxylation.

An advantage of the invention is that the diffusion causes the diffusible material to become saturated with the agent. Some agents can remain present for a substantial time after the agent is introduced into lumen 44. The saturated diffusible material can inhibit development of other infections or inhibit the development of restraining tissue, or both.

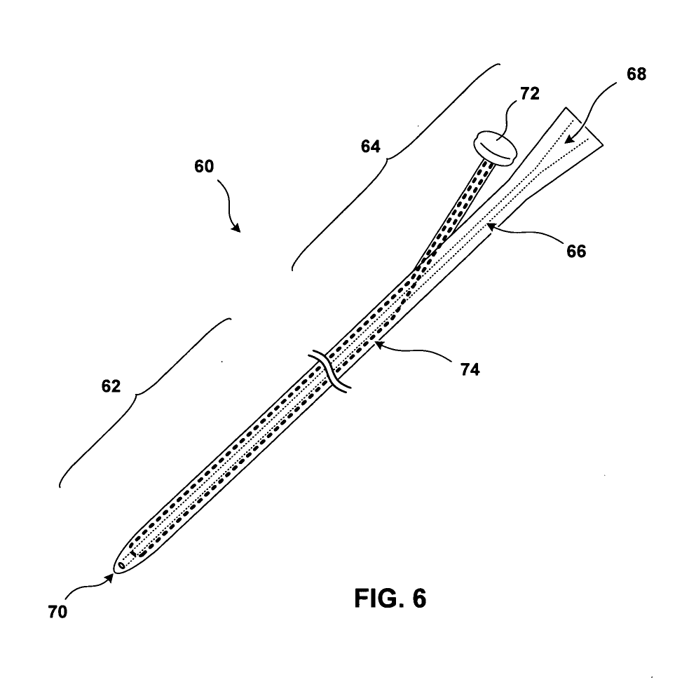

FIG. 6 is a perspective view of an exemplary medical device 60, with phantom lines showing illustrative internal structure. In contrast to implantable medical devices such as medical device 2 in FIGS. 1 and 2, in which the entire device is internal to the body of the patient, exemplary medical device 60 includes an internal element 62 and an external element 64. In the example of FIG. 6, medical device 60 is a catheter that is configured to be partially deployed inside the body of the patient. Internal element 62 at the distal end of medical device 60 is inserted into the body of a patient and is deployed proximate to living cells in the patient. Internal element 62 may be inserted endoscopically through a surgical incision, for example, or may be inserted into a natural anatomical passageway such as a nostril, mouth, urethra, vagina or anus. External element 64 at the proximal end of medical device 60 remains outside the body. Medical device 60 includes a passageway 66, with a proximal opening 68 and a distal opening 70. Passageway 66 can facilitate introduction of fluids into the patient, withdrawal of fluids from the patient, keep a patient's anatomical passageway open, or assist with some other function.

Internal element 62 includes a diffusible material. In some embodiments of medical device 60, the diffusible material covers an underlying structure, such as a metallic or plastic structure that provides rigidity to internal element 62. In other embodiments of medical device 60, medical device 60 is formed principally of or exclusively of the diffusible material.

Medical device 60 includes a port 72 configured to receive an antibiotic agent. Port 72 may comprise any device for receiving an antibiotic agent. In some embodiments of the invention, port 72 may include a reservoir holding the antibiotic agent. For convenience, port 72 is disposed as part of external element 64. Port 72 may likewise be configured to receive a loosening agent. Ordinary use of medical device 60, however, may be unlikely to place internal element 62 inside the body of the patient for a time long enough for restraining tissue to form. For simplicity, medical device 60 will be discussed in terms of receiving an antibiotic agent.

The antibiotic agent that enters port 72 passes into lumen 74, which is in fluid communication with port 72. Lumen 74 extends into internal element 62. The antibiotic agent circulates through lumen 74 and diffuses through the diffusible material to the living cells. Passageway 66, in some embodiments of the invention, is surrounded by a lining (not shown in FIG. 6) that prevents fluids present in passageway 66 from diffusing through the diffusible material, or that prevents antibiotic agents from diffusing into passageway 66. Antibiotic agents diffusing from lumen 74 through the diffusible material of internal element 62 kill harmful organisms that are in contact with medical device 60.

Medical device 60 is not a long-term implant, but medical device 60 may be in place inside the patient for a period of time that would result in a substantial risk of infection. A health care professional may introduce an antibiotic agent into lumen 74 via port 72 every few days, for example, to kill infections proximate to the surface of internal element 62.

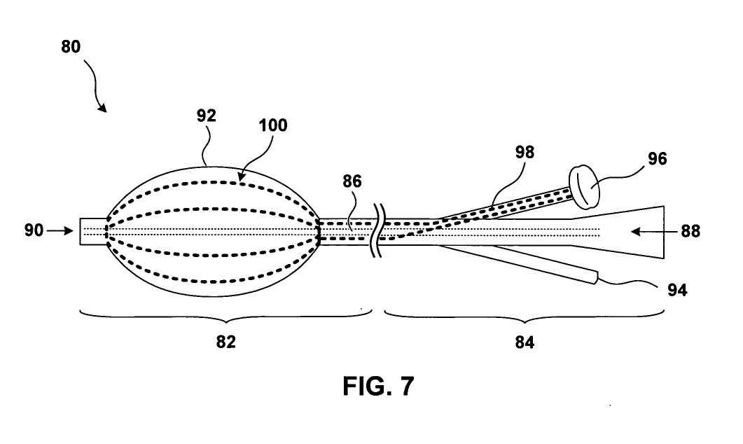

FIG. 7 is a perspective view of another exemplary medical device 80, with phantom lines showing illustrative internal structure. Like medical device 60 in FIG. 6, medical device 80 includes an internal element 82 at the distal end, and an external element 84 at the proximal end. Internal element 82 of medical device 80 is inserted into the body of a patient and is deployed proximate to living cells in the patient, and external element 84 remains outside the body. Medical device 80 optionally includes a passageway 86, with a proximal opening 88 and a distal opening 90. Passageway 86 can facilitate introduction of fluids into the patient, withdrawal of fluids from the patient, keep a patient's anatomical passageway open, or can provide a passageway for additional apparatus such as a probe, an instrument, or an optical fiber.

Internal element 82 includes a diffusible material. In addition, internal element 82 includes a balloon 92, which is shown in an inflated configuration in FIG. 7. Balloon 92 may be formed of biocompatible material that may be elastomeric and diffusible. Balloon 92 may be any shape, and need not be ball-like as shown in FIG. 7. Medical personnel use inflation port 94 to inflate and deflate balloon 92.

Medical device 80 includes a port 96 configured to receive an antibiotic or loosening agent. For simplicity, medical device 80 will be discussed in terms of receiving an antibiotic agent. The antibiotic agent that enters port 96 passes into lumen 98, which is in fluid communication with port 96. Lumen 98 extends into internal element 82. In balloon 92, lumen 98 branches out to become a lumen array 100. The antibiotic agent diffuses through the diffusible material of balloon 92 to the living cells. In some embodiments of the invention, balloon 92 is configured as the principal site of diffusion for medical device 80, such that diffusion occurs at the site of balloon 92 and nowhere else.

Medical device 80 depicted in FIG. 7 can provide antibacterial or antitumoral therapy at targeted sites in the body of the patient. A health care professional steers the distal end of medical device 80 to the site of a chronic localized infection or cancerous tumor, and inflates balloon 92 to bring balloon 92 into close proximity with the infection or tumor. The health care professional introduces an antibiotic agent into port 96, and the antibiotic agent diffuses through balloon 92 to the targeted cells.

Diffusing ROS agents are one kind of many potentially effective agents, and can be useful against infections and tumors. ROS agents such as hydrogen peroxide are effective against bacterial infections, whether aerobic or anaerobic. ROS agents have also been observed to have a cytotoxic effect upon a poorly vascularized tumor, thereby stopping or reducing tumor growth. The health care professional may also select one antibiotic agent to address a bacterial infection and a different antibiotic agent to address a tumor.

Some embodiments of the invention depicted in FIG. 7 can be deployed through a surgical incision, with internal element 82 entering the body and external element 84 remaining outside. Other embodiments can be deployed without surgery.

A variation of the invention depicted in FIG. 7 is fully implantable. In particular, an implantable medical device such as drug pump may be coupled to a balloon with a lumen array, similar to balloon 92 and lumen array 100 shown in FIG. 7. In this variation, the balloon may be deployed proximate to the target cells and inflated. The drug pump may circulate an antibiotic agent in the lumen array, and the antibiotic agent may diffuse through the lumen array to the target cells.

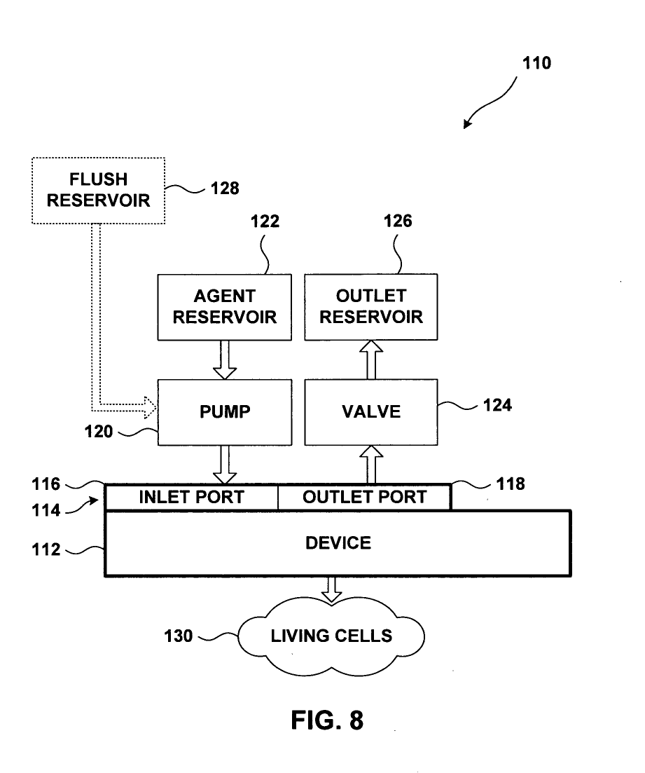

FIG. 8 is a block diagram of a system that can implement the invention. A medical device 112 includes at least one element configured to be deployed proximate to living cells 130 in a patient. Medical device 112 can be fully implantable, or have internal and external elements. Medical device 112 further includes a port 114 configured to receive an antibiotic agent. In the embodiment depicted in FIG. 8, port 114 comprises an inlet port 116 and an optional outlet port 118. Inlet and outlet ports 116, 118 are in fluid communication with one or more lumens or a lumen array (not shown in FIG. 8) in medical device 112. One or more afferent lumens conduct antibiotic agents away from inlet port 116, and one or more efferent lumens conduct the antibiotic agents toward from outlet port 118. Medical device 112 further includes a diffusible material, such that antibiotic agents administered into lumens or a lumen array can diffuse from the lumens or lumen array into the proximate living cells 130.

Pump 120 moves an antibiotic agent from agent reservoir 122. Pump 120 and reservoir 122 may be any kind of pump and reservoir. For example, pump 120 and reservoir 122 can be embodied as a hand-operated syringe, or as a mechanically operated implantable drug pump. There may be implementations of the invention in which the pressure of the antibiotic agent inside the lumens is of importance. Pump 120 can be controlled to produce the desired pressure.

In some embodiments of the invention, outlet port 118 is coupled to a valve 124, which can control the discharge of the antibiotic agent at outlet port 118. When antibiotic agent is introduced into inlet port 116, valve 124 would typically be open to promote circulation of the antibiotic agent through the lumens or lumen array. Once the lumens or lumen array were loaded with the antibiotic agent, valve 124 may be closed to prevent leakage.

After a time, a quantity of the antibiotic agent may have diffused into the surrounding tissues. The supply of antibiotic agent in the lumens or lumen array can be replenished by repeating the loading procedure described above.

An optional outlet reservoir 126 may be provided to receive fluids that discharge from outlet port 118. Substantial quantities of fluid may emerge when, for example, a new or fresh dose of antibiotic agent is introduced with pump 120 and reservoir 122. In some embodiments of the invention, a flush reservoir 128 may hold a flushing liquid, such as saline solution or deionized sterile water, that pump 120 introduces into the lumens or lumen array to flush the antibiotic agent. Outlet reservoir 126 catches the flushed antibiotic agent and flushing liquid.

A plurality of antibiotic agents can be administered via system 110. A first antibiotic agent may be introduced into the lumens or lumen array via inlet port 116 and allowed to diffuse to living cells 130 proximate to device 112. After a time, the lumens or lumen array may be flushed, and a second antibiotic agent may be introduced. In this way, an antibiotic therapy can be tailored to the needs of a particular patient. System 110 can also be adapted to receive a loosening agent from agent reservoir 122.

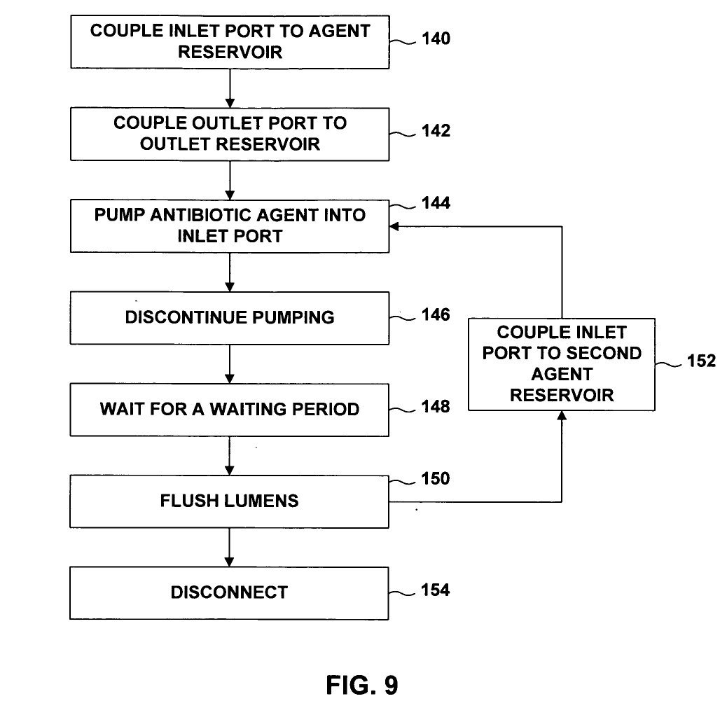

FIG. 9 is a flow diagram that shows an exemplary procedure for use of system 110. A procedure such as depicted in FIG. 9 can be employed whenever a health care professional deems the procedure desirable. A health care professional may employ the procedure to prevent development of an infection, for example, or to treat an existing infection, or to administer therapy such as antitumoral therapy to target tissues. The procedure may also be employed automatically by a medical device. The procedure can be employed with an antibiotic or loosening agent, but will be described in terms of an antibiotic agent.

The health care professional couples agent reservoir 122 to inlet port 116 (140). In some procedures, the coupling may take place without the creation of an incision, such as is depicted in FIG. 2. In other procedures, the health care professional may be deem it advantageous to obtain access to the internal element through an incision. In further procedures, inlet port 116 may be external to the body of the patient. FIGS. 6 and 7 show exemplary instruments that include an external port, and the health care professional may couple the agent reservoir to an external inlet port without further invasion of the body. Optionally, the health care professional couples outlet reservoir 126 to outlet port 118 (142). In the case of a medical device, agent reservoir 122 and inlet port 116 may have been previously coupled to one another, and outlet reservoir 126 and outlet port 118 may have been previously coupled as well.

The health care professional or the medical device loads the antibiotic agent into inlet port 116 with pump 120 (144). As a result, the lumens in the internal element receive the antibiotic agent. Pumping may be discontinued (146) using any practical criteria. In one example, a health care professional loading the antibiotic agent with a syringe discontinues loading when the syringe is empty. In another example, a health care professional discontinues loading when the antibiotic agent discharges from outlet port 118. In a further example, a medical device discontinues pumping when the fluid pressure in the lumens reaches a target pressure.

Optionally, the health care professional or medical device waits for a waiting period (148). During the waiting period, the antibiotic agent in the lumens diffuses through the diffusible material to the nearby living cells. The length of the waiting period depends upon factors such as the diffusion rate, the antibiotic agents being used, and the nature of the therapy. In a typical case in which thirty percent hydrogen peroxide diffuses through a tube of biocompatible silicone, the waiting period may be about one hour.

After the waiting period expires, the lumens may be flushed with a flushing liquid from flushing reservoir 128 (150). Thereafter a second agent reservoir can be coupled to inlet port 116 (152), and the loading procedures may be repeated. The second agent reservoir may hold the same antibiotic agent or a different agent. The reservoirs may be disconnected from the respective ports to complete the procedure (154). In the instances in which access to the medical device has been obtained through surgery, the ports may be capped if appropriate, and the surgical opening is closed.

The procedure depicted in FIG. 9 is illustrative, and the invention is not limited to the procedure depicted therein. In some cases, flushing may be omitted. For example, after the waiting period, an additional quantity of the antibiotic agent may be pumped into the inlet port without intermediate flushing. There may also be cases in which introduction of a second agent is deemed unnecessary. Further, although a pump may be a very effective tool for introducing the antibiotic agent into the lumens, the invention supports introduction of the antibiotic agent by an instrument other than a pump. In some embodiments, the antibiotic agent may be gravity fed to the lumens from a drip bag, for example, or introduced from an agent reservoir under pressure.

The illustrative procedure depicted in FIG. 9 can be employed in a prophylactic or preventative fashion. For example, the procedure can be employed to prevent the development of infections on the surfaces of the internal elements of medical devices. The procedure can also be applied in a therapeutic fashion, to address existing infections or tumors.

FIG. 10 is a conceptual diagram illustrating an in vitro testing system 160 with an antibiotic agent. Testing system 160 includes a culture plate 162, which serves as a test platform. As shown in FIG. 10, the test platform contains a growth medium 164 that supports a test microorganism culture 166, which serves as an indicator. A experimental tube 168 made at least in part of diffusible material is deployed in plate 162 proximate to microorganisms 166. The diffusible material is configured to pass the test agent to the indicator by diffusion. Experimental tube 168 may be wholly or partially embedded in growth medium 164, or may rest atop growth medium 164. Experimental tube 168 includes a lumen configured to receive a test antibiotic agent.

FIG. 10 shows a path of the antibiotic agent through testing system 160. A pump 170 pumps the antibiotic agent from a reservoir 172 into the lumen of tube 168. A valve 174 may be closed to prevent leakage or other discharge of the antibiotic agent from tube 168, and an outlet reservoir 176 may be provided to collect fluid that discharges from tube 168. In some embodiments, pump 170, valve 174, or both are electronically controlled such that the quantity of antibiotic agent in tube 168 is known, such that the pressure of antibiotic agent in tube 168 is known, or such that the flow rate of antibiotic agent in tube 168 is known. The effect of the antibiotic agent on microorganisms 166, if any, can be observed using conventional observational techniques, such as optical or microscopic examination. In this way, the effect of the antibiotic agent on microorganisms 166 serves as an indicator of diffusion of the test agent through the diffusible material.

The system shown in FIG. 10 is useful for testing elements that may be used in medical devices such as devices depicted in FIGS. 1-7. Such testing supports assessing the effectiveness of an embodiment of the invention prior to use in vivo. In particular, system 160 can be used to conduct experiments pertaining to the diffusion of the antibiotic agent through experimental tube 168. Results of the experiments can support a selection of one diffusible material over another, for example. One diffusible material may be selected over another because the antibiotic agent diffuses through the selected diffusible material at a desirable rate. Test system 160 can reveal whether antibiotic agent diffuses through a diffusible material to quickly, or not readily enough. Test system 160 can further reveal whether antibiotic agent retains potency following diffusion.

Test system 160 can further be employed to test the geometry of the diffusible material. It may be discovered, for example, that the antibiotic agent diffuses well through the diffusible material when the walls of the diffusible material have a particular range of thicknesses.

In some embodiments of test system 160, agent reservoir 172 may supply the test antibiotic agent two to two or more similar plates simultaneously. Experimental tubes of different configurations, having different geometries or being made of different diffusible materials, may be deployed in the respective plates, proximate to the respective test cell or bacterial cultures.

The invention also supports a “control” plate, in which a tube deployed in one plate includes no diffusible material. The antibiotic agent introduced into the lumen of the tube in the “control” plate would be unable to diffuse from the lumen to the test cell culture. Alternatively, the lumen of the tube of the “control” plate may be disconnected from the agent reservoir, and may receive a fluid such as deionized water in place of the antibiotic agent.

Testing system 160 can be adapted to analyze experimental tube 168 with a test loosening agent. Although the test platform can include a test microorganism culture that reacts to the presence of the loosening agent, it is not necessary that a microorganism culture be used as an indicator. The test platform can also include one or more nonliving indicators, such as a chemical indicator.

The invention may realize one or more advantages. Various embodiments of the invention, particularly medical devices that are deployed inside a patient for extended periods of time, can be protected from infection by applying the techniques of the invention. Periodic loading of antibiotic agents can serve as a preventative measure against infection. In addition, the techniques of the infection are effective against existing infections, including those that would be shielded from antibiotic agents in the body systems by biofilm, tissue encapsulation or other barriers. When the antibiotic agents are delivered according to the invention, side effects are expected to be low, because certain healthy, well-vascularized tissues are generally not adversely affected by concentrations of some antibiotic agents.

In comparison to antibiotic agents administered to the whole patient, such as antibiotics administered orally or by injection, the antibiotic agents administered according to the invention can be targeted. The antibiotic agents administered according to the invention diffuse through diffusible material to proximate living cells. In this way, the antibiotic agents are targeted to living cells that are proximate to an internal element of a medical device. Because the antibiotic agents are targeted, the effective concentrations need not be as high as concentrations administered to the whole patient.

A further potential advantage of targeting is that a medical device may be deliberately moved proximate to target cells, and antibiotic agents may be administered by diffusion to those target cells. As illustrated by the device shown in FIG. 7, for example, an element such as a balloon can be used to bring the diffusible material proximate to the target cells.

In addition, the invention can realize the advantage of improving surgical removal of implanted devices. Introduction of a loosening agent by diffusion thorough a diffusible material can help prevent the development of restraining tissue that could impede access to or removal of the device. Introduction of a loosening agent can also disengage the device from the restraining tissue, facilitating extraction. In addition, some loosening agents can be antibiotic agents, and vice versa. Hydrogen peroxide is one example of an agent that can serve as both an antibiotic agent and as a loosening agent.

Various embodiments of the invention have been described. The invention is not limited to those particular embodiments, but includes other embodiments as well, including modifications made to the described embodiments. For example, the invention encompasses embodiments in which a medical device includes multiple internal elements, multiple diffusible materials, multiple agents, or combinations thereof. The invention also supports embodiments in which some agents are administered by techniques in addition to diffusion. For example, a single patient may receive a first antibiotic agent by mouth, and a second antibiotic agent by diffusion. These and other embodiments are within the scope of the following claims.

1. A medical device comprising:
an element configured to be deployed proximate to living cells in a patient, and
a port configured to receive an antibiotic agent,
wherein the element comprises a diffusible material configured to be in contact with the living cells,
wherein the diffusible material comprises at least one lumen in fluid communication with the port, and
wherein the diffusible material is configured to diffuse the antibiotic agent in the lumen to the living cells.

2. The device of claim 1, wherein the diffusible material is configured to diffuse an antibacterial agent.

3. The device of claim 1, wherein diffusible material is configured to diffuse a cytotoxic agent.

4. The device of claim 1, wherein the diffusible material is configured to diffuse at least one Reactive Oxygen Species.

5. The device of claim 4, wherein the Reactive Oxygen Species comprises hydrogen peroxide.

6. The device of claim 1, wherein the diffusible material is configured to diffuse a loosening agent configured to disengage the device from restraining tissues.

7. The device of claim 1, wherein the port is configured to couple to a reservoir containing the antibiotic agent.

8. The device of claim 1, wherein the port comprises:
an inlet port that receives the antibiotic agent and directs the antibiotic agent to an afferent lumen; and
an outlet port that receives the antibiotic agent from an efferent lumen in fluid communication with the afferent lumen and discharges the antibiotic agent.

9. The device of claim 8, further comprising an outlet reservoir coupled to the outlet port configured to receive the discharged antibiotic agent.

10. The device of claim 1, further comprising a plurality of lumens coupled to the port to distribute the antibiotic agent within the element.

11. The device of claim 10, wherein the lumens are arranged in an array.

12. The device of claim 1, wherein the device is configured to be implantable in a human body.

13. The device of claim 1, wherein the device comprises at least one of an implantable pacemaker, an implantable defibrillator, an implantable drug pump, a catheter, and a patient monitor.

14. The device of claim 1, wherein the element comprises a balloon configured to be inflated to move the lumen.

15. The device of claim 1, wherein the diffusible material comprises at least one of biocompatible silicone, polyethylene or polyurethane.

16. The device of claim 1, wherein the element is an internal element; the device further comprising an external element configured to be deployed outside a body of the patient.

17. The device of claim 1, wherein the element configured to be deployed proximate to the living cells is configured to jacket a structural element of a medical device configured to be deployed proximate to living cells in a patient

18. The device of claim 17, wherein the diffusible material comprises an elastomer configured to stretch to allow the device to receive the medical device, and to contract to be held in place with respect to the medical device.

19. The device of claim 1, wherein the port is a first port and the lumen is a first lumen, the device further comprising:
a second port configured to receive the antibiotic agent,
wherein the diffusible material comprises at least one second lumen in fluid communication with the second port.

20. A method comprising:
introducing an antibiotic agent into a port of a medical device, wherein the medical device includes at least one element configured to be deployed proximate to living cells in a patient; and
diffusing the antibiotic agent to the cells via a diffusible material in contact with the living cells,
wherein the diffusible material comprises at least one lumen in fluid communication with the port.

21. The method of claim 20, further comprising introducing a flushing liquid into the port of the medical device to flush the antibiotic agent from the lumen.

22. The method of claim 20, further comprising:
waiting for a waiting period; and
introducing additional antibiotic agent into the port of the medical device.

23. The method of claim 20, wherein the antibiotic agent is a first antibiotic agent, the method further comprising:
waiting for a waiting period; and
introducing a second antibiotic agent different from the first antibiotic agent into the port of the medical device.

24. The method of claim 20, further comprising:
inserting the element into the patient; and
inflating a balloon coupled to the element, wherein the balloon is formed at least partially from the diffusible material.

25. The method of claim 20, wherein the port comprises an inlet port, the method further comprising coupling an agent reservoir to the inlet port.

26. The method of claim 20, wherein the port comprises an outlet port, the method further comprising coupling an outlet reservoir to the outlet port.

27. The method of claim 20, wherein the antibiotic agent comprises at least one of an antibacterial agent, a cytotoxic agent, a Reactive Oxygen Species or a loosening agent.

28. A device comprising:
an elongated primary core having an axis surrounded by a diffusible material having an exterior surface;
a first lumen in the diffusible material configured to conduct an antibiotic agent in a first direction; and
a second lumen in the diffusible material configured to conduct the antibiotic agent in a second direction different from the first direction,
wherein the diffusible material is configured to diffuse the antibiotic agent from at least one of the lumens to the exterior surface.

29. The device of claim 28, wherein the primary core comprises an electrical lead.

30. The device of claim 28, wherein the primary core comprises a passageway configured to receive at least one of a fluid, a probe, an instrument, or an optical fiber.

31. The device of claim 28, further comprising a lining proximate to the primary core, the lining configured to inhibit diffusion of the antibiotic agent from at least one of the lumens to the primary core.

32. The device of claim 28, wherein the diffusible material is configured to diffuse at least one of an antibacterial agent, a cytotoxic agent, or a Reactive Oxygen Species or a loosening agent.

33. A device comprising:
a port configured to receive an antibiotic agent; and
at least one lumen in fluid communication with the port; and
a diffusible material,
wherein the diffusible material is configured to diffuse the antibiotic agent in the lumen to living cells in a patient, and
wherein the device is configured to jacket at least part of a medical device.

34. The device of claim 33, wherein the diffusible material comprises at least one of biocompatible silicone, polyethylene or polyurethane.

35. The device of claim 33, wherein the diffusible material comprises an elastomer configured to stretch to allow the device to receive the medical device, and to contract to be held in place with respect to the medical device.

36. The device of claim 33, further comprising a set of lumens arranged in an array.

37. A system comprising:
a test platform configured to support an indicator; and
an experimental tube made at least in part of diffusible material deployed in the test platform proximate to the indictor;
wherein the experimental tube comprises a lumen configured to receive and to pass a test agent by diffusion, and
wherein the test agent comprises at least one of a test antibiotic agent or a test loosening agent.

38. The system of claim 37, wherein the test platform comprises a culture plate containing a growth medium configured to support a test cell culture, and wherein the indicator comprises the test cell culture.

39. The system of claim 37, further comprising an agent reservoir configured to supply the test agent to the lumen.

40. The system of claim 39, further comprising a pump configured to move the test agent from the agent reservoir to the lumen.

41. The system of claim 37, further comprising an outlet reservoir configured to collect the test agent that discharges from the lumen.

42. The system of claim 37, further comprising a valve configured to control discharge of the test agent from the lumen.

43. A method comprising:
introducing a test agent into a lumen of an experimental tube deployed in a test platform, wherein the test agent comprises at least one of a test antibiotic agent or a test loosening agent, and wherein the experimental tube deployed proximate to an indicator in the test platform; and
observing an effect upon the indicator,
wherein the experimental tube is made at least in part of diffusible material, and
wherein the diffusible material is configured to pass the test agent to the indicator by diffusion.

44. The method of claim 43, wherein the experimental tube is a first experimental tube, the test platform is a first test platform, and the indicator is a first indicator, the method further comprising:
introducing the test agent into a lumen of a second experimental tube deployed in a second test platform, the second experimental tube deployed proximate to a second indicator in the second culture plate; and
observing an effect upon the second indicator.

45. The method of claim 44, wherein the diffusible material is a first diffusible material, and wherein the second experimental tube is made at least in part of a second diffusible material different from the first diffusible material.

46. The method of claim 44, wherein the first experimental tube has a first geometry and second experimental tube has a second geometry.

47. The method of claim 43, wherein the test platform comprises a culture plate containing a growth medium configured to support a test cell culture, and wherein the indicator comprises the test cell culture.

48. A medical device comprising:
an element configured to be deployed proximate to restraining tissues in a patient, and
a port configured to receive a loosening agent configured to disengage the medical device from the restraining tissues,
wherein the element comprises a diffusible material configured to be in contact with the restraining tissues,
wherein the diffusible material comprises at least one lumen in fluid communication with the port, and
wherein the diffusible material is configured to diffuse the loosening agent in the lumen to the restraining tissues.

49. The device of claim 48, wherein the diffusible material is configured to diffuse an antibiotic agent.

50. The device of claim 48, wherein the diffusible material is configured to diffuse at least one Reactive Oxygen Species.

51. The device of claim 50, wherein the Reactive Oxygen Species comprises hydrogen peroxide.

52. The device of claim 48, wherein the port comprises:
an inlet port that receives the loosening agent and directs the loosening agent to an afferent lumen; and
an outlet port that receives the loosening agent from an efferent lumen in fluid communication with the afferent lumen and discharges the loosening agent.

53. The device of claim 48, further comprising a plurality of lumens coupled to the port to distribute the loosening agent within the element.

54. The device of claim 48, wherein the device is configured to be implantable in a human body.

55. The device of claim 48, wherein the device comprises at least one of an implantable pacemaker, an implantable defibrillator, an implantable drug pump, a catheter, and a patient monitor.

56. The device of claim 48, wherein the diffusible material comprises at least one of biocompatible silicone, polyethylene or polyurethane.

57. The device of claim 48, wherein the element configured to be deployed proximate to the restraining tissue is configured to jacket a structural element of a medical device configured to be implantable in a patient

58. A method comprising:
introducing a loosening agent configured to disengage a medical device from restraining tissues in a patient into a port of the medical device; and
diffusing the loosening agent to the restraining tissues via a diffusible material in contact with the restraining tissues,
wherein the diffusible material comprises at least one lumen in fluid communication with the port.

59. The method of claim 58, further comprising:
waiting for a waiting period; and
introducing additional loosening agent into the port of the medical device.

60. The method of claim 58, wherein the antibiotic agent comprises at least one of a Reactive Oxygen Species.

61. A device comprising:
a port configured to receive a loosening agent configured to disengage a medical device from restraining tissues in a patient;
at least one lumen in fluid communication with the port; and
a diffusible material,
wherein the diffusible material is configured to diffuse the loosening agent in the lumen to the restraining tissues, and
wherein the device is configured to jacket at least part of the medical device.

62. The device of claim 61, wherein the diffusible material comprises at least one of biocompatible silicone, polyethylene or polyurethane.

63. The device of claim 61, wherein the diffusible material comprises an elastomer configured to stretch to allow the device to receive the medical device, and to contract to be held in place with respect to the medical device.

64. The device of claim 61, further comprising a set of lumens arranged in an array.
